(19) United States
(12) Patent Application Publication
(10) Pub. No.: US 2006/0106415 A1
(43) Pub. Date: May 18, 2006

(54) Apparatus to facilitate implantation

(76) Inventor: Shlomo Gabbay, Short Hills, NJ (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 10/987,605

(22) Filed: Nov. 12, 2004

(51) Int. Cl. A61M 29/00

(52) U.S. Cl. 606/198

(57) ABSTRACT

One introducer apparatus may include a flange and a sleeve having a sidewall that extends longitudinally from the flange and terminates in a distal end portion. At least one adjustable opening is operatively associated with the distal end portion. The adjustable opening is moveable from a substantially closed condition to an open condition, which permits substantially free movement of an elongate member axially through a passage defined by the sidewall. An implanter can be utilized in combination with the introducer apparatus to facilitate implanting an article in a patient.

### TECHNICAL FIELD

The present invention relates to implantation of a prosthesis and, more particularly, to an apparatus and method to facilitate implantation.

### BACKGROUND

Various types of prostheses have been developed and corresponding approaches are utilized to implant prostheses in both human and non-human patients. It is well known to utilize annuloplastic rings, stents, heart valves and other implantable devices for helping improve cardiac operation valves in human patients. Oftentimes implantation of a prosthesis requires passage of the prosthesis through surrounding tissue, such as when the prosthesis is being implanted at a site located within an organ or within a protective covering or sheath (e.g., skin muscle). For example, to surgically implant a heart valve prosthesis into a patient, the patient typically is placed on cardiopulmonary bypass during a complicated, but common, open chest and usually open-heart procedure. Such procedures tend to be invasive to the patient and, in certain situations, may present great risk.

### SUMMARY

The present invention relates generally to an apparatus to facilitate implantation of an article, such as a prosthesis, an implant or other device.

One aspect of the present invention provides an introducer apparatus that includes a flange and a sleeve having a sidewall that extends longitudinally from the flange and terminates in a distal end portion. At least one adjustable opening is operatively associated with the distal end portion. The adjustable opening is moveable from a substantially closed condition to an open condition, which permits substantially free movement of an elongate member axially through a passage defined by the sidewall.

Another aspect of the present invention provides an implantation system that includes an elongate barrel that terminates in an open end. The elongate barrel may form part of an implanter. An introducer apparatus has a first end spaced apart from a distal end by an elongated sidewall portion. The sidewall portion provides a passage that extends axially through the introducer apparatus, the passage being dimensioned and configured for receiving the barrel. The sidewall portion includes a tapered distal end portion that is moveable from a substantially closed condition to an open condition in response to axial movement of the barrel from the first end and at least partially through the distal end such that barrel engages an interior of the distal end portion.

Yet another aspect of the present invention provides an implantation system that includes means for implanting an article as well as means for providing a passage through an opening formed in tissue of a patient to guide the means for implanting toward an implantation site. The means for providing has an end portion that is moveable from a closed condition to an open condition in response to axial movement of the means for implanting through the means for providing to facilitate positioning an end of the means for implanting near the implantation site. The implantation system also includes means for discharging the article from the means for implanting.

### BRIEF DESCRIPTION OF THE DRAWINGS

FIG. 1 depicts an example of an introducer apparatus in accordance with an aspect of the present invention.

FIG. 2 depicts an example of the introducer apparatus of FIG. 1 demonstrating operation thereof in conjunction with an elongate member in accordance with an aspect of the present invention.

FIG. 3 depicts an example of an implanter apparatus for implanting a prosthesis in accordance with an aspect of the present invention.

FIG. 4 depicts an example of an introducer apparatus being inserted at an in the aorta of a heart in accordance with an aspect of the present invention.

FIG. 5 depicts an example of a valve being implanted at an aortic position in accordance with an aspect of the present invention.

FIG. 6 depicts an example of an introducer apparatus being inserted near an apex of a heart in accordance with an aspect of the present invention.

FIG. 7 depicts an example of a valve being implanted at the pulmonic position in accordance with an aspect of the present invention.

### DESCRIPTION

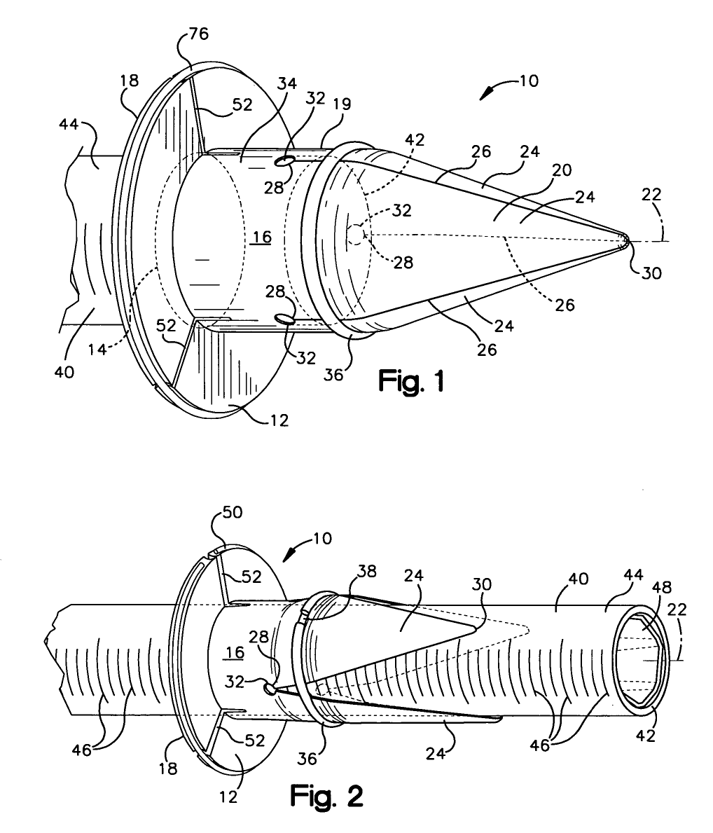

FIGS. 1 and 2 depict an example of an introducer apparatus 10 that can be implemented in accordance with an aspect of the present invention. The introducer apparatus 10 includes a flange 12. While the flange 12 is illustrated as a complete annular flange, those skilled in the art will understand and appreciate that the flange 12 can be implemented in a variety of shapes (e.g., rectangular hexagonal, etc.) and that flange 12 need not extend in a complete annular structure. For example, the flange 12 can be implemented as substantially co-planner tabs, a rim, as well as a curved or c-shaped flange portion or as a thicker sidewall portion of the apparatus 10.

In the example of FIG. 1, the flange 12 includes an inner periphery 14 that defines an opening into an associated sleeve 16. While the inner periphery is 14 generally circular in the examples of FIGS. 1 and 2, it can have other shapes. The flange 12 also includes an outer periphery 18 spaced apart from the inner periphery 14 by an associated intermediate portion thereof.

The sleeve 16 has a sidewall 19 that extends longitudinally from the flange 12 and terminates in a distal end portion 20. A central axis 22 extends through a center of the sleeve 16 and through the opening defined by the inner periphery 14 of the flange 12. An adjustable opening is operatively associated with the distal end portion. The adjustable opening includes means movable from at least a closed condition (FIG. 1) to an open condition (FIG. 2) to permit substantially free movement of an article 44, such as an implanter or trocar, axially through a passage defined by the sidewall 19.

By way of example, the adjustable opening in the distal end portion 20 can include two or more jaw members 24 that are movable in a generally radially direction relative to the axis 22 between the open and closed conditions. In the example of FIG. 1, the distal end portion 20 includes a plurality of three jaw members 24 positioned in closed condition. Longitudinally extending side edges of adjacent jaw members 24 define longitudinally extending slits 26. The longitudinally extending slits 26 extend through the sidewall 19 from a first position 28, which is located proximal to the flange 12, to intersect an opposed end 30 of the distal end portion 20 to separate the jaw members 24. An aperture 32 further may extend through the sidewall 19 of the sleeve at the first position 28 of each of the respective slits 26. The respective apertures 32 operate as hinges to facilitate the generally radial movement of the respective jaw members 24 from the closed condition to the open condition. Other structure, such as hinges, can also be employed to provide for moveability of the jaw members 24. Additionally, one of the slits might extend completely through the sidewall 19 as well as through the flange 12 to accommodate differently sized elongate members within the passage. Thus, the sidewall can be generally cylindrical (such as shown in FIGS. 1 and 2, or the sidewall can be generally C-shaped.

In the example of FIG. 1, the sleeve 16 can include an intermediate portion 34 located between the flange 12 and the distal end portion 20. The intermediate portion 34 of the sleeve 16 can have a generally right circular cylindrical cross section that extends a predetermined length of from the flange substantially coaxial with the axis. The remainder of the sleeve 16, corresponding generally to the distal end portion 20, has a generally conical shape when in the closed condition depicted in FIG. 1. As an example, the slits 26 extend from the end 30 of the distal end portion 20 through the sidewall 19 to the position 28 which can be located between the respective ends of the intermediate portion 34 of the sleeve 16. Those skilled in the art will appreciate that the first portion 34 and distal end portion 20 may have other shapes than as shown and described herein.

The introducer apparatus 10 can be formed of a variety of materials including metals, alloys polymers and/or composites, although it should be made of a material that is considered biocompatible or that can be made sufficiently biocompatible for at least temporary insertion into a desired tissue of a patient. Additionally, the flange 12 and sleeve 16 can be formed of the same or different materials. When formed of the same material, such as a plastic or thermoplastic material, the entire implanter apparatus can be injection molded from a common material. Different materials can also be utilized for the various parts of the apparatus 10.

The sidewall 19 of the sleeve 16 has a thickness that can be selected according to the material utilized for the sleeve to enable desired movement of the distal end portion to between the opened and closed conditions thereof. For instance, the sleeve 16, or at least the jaw members 24 thereof, can be formed of a flexible or pliant material to facilitate movement of the jaw members from the closed to open condition, such as upon insertion of an elongate member therethrough. The materials utilized might also be an in elastically deformable material so that jaw members 24 may remain in a substantially open condition after removal of the elongate device. No amount of resilience or memory of the jaw members or memory of the material is required for implementing the introducer apparatus 10.

Additionally, the introducer apparatus 10 can include means, such as an annular structure (e.g., a ring) 36, to inhibit movement of the distal end portion from the closed condition to the open condition. The annular structure 36 also inhibits relative movement of the elongate member through the introducer apparatus 10. In FIGS. 1 and 2, the structure 36 is depicted as a ring mounted around an exterior of the distal end portion 20 of the sidewall 19 of the introducer apparatus 10. Those skilled in the art will understand and appreciate other shapes (C-shaped or U-shaped) and configurations of structures that can be applied to the sidewall 19 to implement the functions described herein as being performed by the structure 36.

In the example of FIG. 1, the annular structure 36 applies a radially inward force to help hold the jaw members 24 in the substantially closed position. As a result, the elongate member generally is only insertable until the end 42 of the elongate member 40 engages the interior surface of the distal end portion. By inhibiting insertion of the elongate member through the apparatus, the annular structure 36 facilitates insertion of the combination of the elongate member and introducer apparatus 10 into an anatomical structure of a patient. The ring structure 36 also operates to maintain the distal end portion and jaw members 24 in a generally conical arrangement as it is urged into an anatomical structure for implantation.

As shown in FIG. 2, the structure 36 can rest in a groove (or in one or more slots or notches) 38 to maintain the structure 36 at a desired axial position along the exterior of the sidewall 19 of the introducer apparatus 10. The structure 36 can be fixed (e.g., by an adhesive or ultrasonic welding) to a portion of the sidewall 19 or it can hold its position due to frictional forces. The structure 36 can be formed of a plastic, metal, rubber (e.g., a rubber-band-like or O-ring structure) or other material that can be employed to apply radially inward force to the sidewall 19. Thus, the structure 36 can be applied to urge the jaw members in a closed condition, as shown in FIG. 1.

FIG. 2 further depicts the elongate member 40 inserted through the passage of the apparatus 10 such that the respect jaw members 24 are in an open condition. As mentioned above, the jaw members 24 can be urged into the open condition by inserting the elongate member 40 through the opening associated with the inner periphery 14 of the flange 12 and axially through the passage defined by the sidewall 19 of the introducer apparatus 10. In the example of FIG. 2, the elongate member 40 has been inserted within the passage such that a distal end 42 of the elongate member protrudes beyond the end 30 of the distal end portion 20.

To facilitate insertion of the elongate member 40 through the distal end portion 20 of the introducer apparatus, as shown in FIG. 2, the radially inward force being applied by the structure 36 can be removed from the sidewall 19, such as by cutting or removing the part of the structure 36. The removal can be made through a slit or slot 52 formed in the flange 12 or by providing some mechanism for otherwise breaking the radially inward force applied thereby. When the structure 36 is cut, for example, the jaw members 24 can be more easily urged into their open condition so that the elongate member 40 can substantially freely move through the passage defined by the interior of the introducer apparatus 10.

The jaw members 24 thus can be urged into their open condition in response to an exterior surface 44 of the elongate member 40 engaging an interior portion of the sidewall 19, which engagement causes the jaw members 24 to deflect outwardly away from the central axis 22. The material employed for the jaw members 24 can exhibit resilience or some shape memory so that the jaw members return approximately to the closed condition after the elongate member 40 has been withdrawn from the passage defined by the sidewall 19. Alternatively, the jaw members, depending on the material, might be inelastically deformable to remain in a generally open condition upon removal of the elongate member 40.

In the example of FIG. 2, the elongate member 40 is in the shape of a cylindrical barrel. Those skilled in the art will understand and appreciate various shapes and configurations of elongate members can be utilized in combination with introducer apparatus 10. For example, the elongate member 40 can correspond to a portion of an implanter in which a prosthesis (e.g., heart valve, annuloplasty ring, stents, etc.) or other device, indicated schematically at 48, can be positioned for implantation. Alternatively, the elongate member 40 might be a trocar that is employed to provide a passage for accessing (e.g., laparoscopically, arthroscopically, endoscopically, or otherwise) anatomical features within a patient.

An exterior surface 44 of the elongate member 40 can also include indicia 46 that can be utilized to ascertain the length of the elongate member that has been inserted through the introducer apparatus 10. For instance, a proximal end of the introducer apparatus 10 (e.g., the inner periphery 14 of the flange 12) can align with a circumferentially extending indicia 46 to indicate a measurement as to how far the distal end 42 of the elongate member 40 has been inserted through the introducer apparatus, such as a distance beyond the flange 12. This can be used, for example, to position the end 42 of the elongate member 40 accurately relative to anatomical structures within the patient, such as in an organ or other implantation site.

Additionally or alternatively, the introducer apparatus 10 can include another structure 50 applied to the annular flange 12 to inhibit movement of the elongate member 40 axially through the passage defined by the sidewall 19. In the example of FIGS. 1 and 2, the structure 50 is depicted as a ring (e.g., an O-ring) applied to and engaging the outer periphery 18 of the flange 12. To facilitate holding the structure 50 to the flange 12, the outer periphery 18 can be recessed or include a groove around the flange.

One or more slits (or notches) 52 can also extend through the flange 12 to provide an adjustable cross-sectional dimension for the flange. The slits 52, for example, can extend at least from the outer periphery 18 to the inner periphery 14, and may further extend along a proximal part of the sidewall 19, such as shown in FIGS. 1 and 2. Thus, by applying the structure 50 to the outer periphery 18 of the flange 12, the slits 52 can be urged toward a closed condition (FIG. 1) so that the inner periphery 14 of the flange 18 exerts radially inward force along an exterior of the elongate member 40. This radially inward force (while the structure 50 is applied) inhibits axial movement of the introducer apparatus 10 relative to the elongate member 40. As a result, manipulation and insertion of the combination of the elongate member 40 and introducer apparatus 10 into anatomical structures can be facilitated, such as for implanting a prosthesis or other device. After the introducer apparatus 10 has been inserted so that the flange engages the structure, the structure 50 can be cut or removed from the flange (as shown in FIG. 2) to remove the radially inward force being applied. Thus, when the force being applied by the structure 50 has been removed, the elongate member 40 can move substantially freely through the apparatus 10 to facilitate positioning the distal end 42 at or near the desired implantation site.

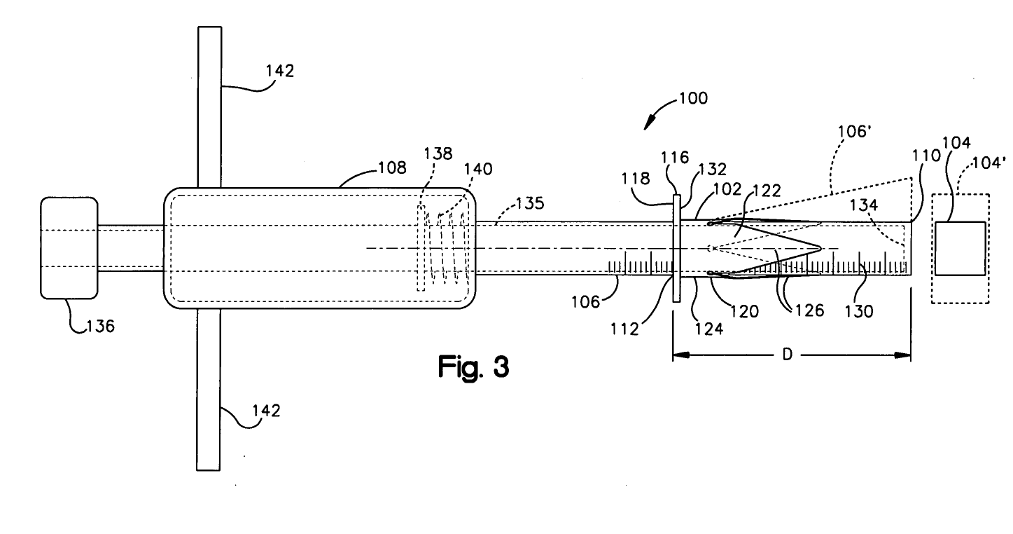

FIG. 3 illustrates an example of an implanter 100 in combination with an introducer apparatus 102 in accordance with an aspect of the present invention. The implanter/introducer combination 100, 102 can be employed for to facilitate low-invasive implantation of an article 104. Those skilled in the art will understand and appreciate various types of prostheses as well as other types of implantable devices that might be implanted using the implanter/introducer combination 100, 102 and, further that modifications to the implanter and/or introducer may be needed depending on the implantation site and the type of prosthesis or device 104.

In the example of FIG. 3, the implanter 100 includes an elongated cylindrical barrel 106 that extends from a body portion 108 and terminates in an open end 110. The barrel 106 has an inner diameter that may vary according to the type of article 104 as well as the dimension and configuration of the article being implanted. The introducer apparatus 102 can be slidably mounted for movement along an exterior of the barrel 106, such as by insertion of the end 110 of the barrel through an opening at a proximal end 112 of the introducer apparatus. The introducer 102 can be substantially similar to that shown and described with respect to FIGS. 1 and 2.

Briefly stated, the introducer apparatus 102 includes a flange portion 116 at the proximal end 112 of the introducer apparatus and a sidewall portion 120 extending longitudinally from the flange portion. The introducer apparatus terminates in a distal portion 122 spaced apart from the flange by an intermediate portion 124. The distal portion 122 includes one or more members, such as jaw members 126, which are moveable relative to each other. The jaw members 126 can be moveable at least from a closed condition to an open condition and, depending on the material, also from the open condition back to the closed condition.

In the example of FIG. 3, the introducer apparatus 102 is slidably positioned for movement along the barrel 106 of the implanter 100. The introducer apparatus 102 may be removable from barrel 10. Alternatively, the end 110 of the barrel 106 can include a retaining feature (e.g., a rib, a tab, thicker sidewall portion, etc. (not shown)) to help prevent removing the introducer apparatus 102 completely from the barrel.

Similar to the example of FIG. 2, the introducer apparatus 102 is depicted in FIG. 3 in an opened condition, in which an exterior surface of the barrel 106 engages in inner surface of the sidewall 120 introducer apparatus. In particular, the inner surface of the sidewall 120 engages the exterior surface of the barrel 106. The engagement between the barrel 106 and the introducer apparatus 102 urges the respective jaws 126 of the introducer apparatus to the open condition. In the open condition, the distal end portion 122 of the introducer apparatus 102 has a generally crown-like appearance, in which adjacent side edges of the respective jaw members 126 are spaced apart from each other in generally V-shaped voids.

As described with respect to FIG. 2, the barrel 106 of the implanter 100 can include indicia 130 disposed along the exterior surface of the barrel between the distal end 110 of the barrel. The indicia 130 can be printed or otherwise marked onto the barrel as a scale or ruler. In this way, the proximal end 112 of flange portion 116 can align with adjacent indicia 130 to identify a measure of the distance indicated at D between the flange portion 116 and the distal end 110 of the barrel 106. The scale or ruler markings that form the indicia 130 further can be adjusted to accommodate for the thickness of the flange portion 116, such that the measurement can correspond to the distance from a distal side of the flange 132 to the end 110 of the barrel 106.

In FIG. 3, the prosthesis or device 104 is illustrated as being discharged from the implanter 100. For example, the implanter 100 includes a plunger or other means 134 for discharging the prosthesis or device 104 from the end 110 of the barrel 106. The implanter 100 can include a flange or other feature 138 that operatively connected with the plunger 134. A biasing element (e.g., one or more springs) 140 is operative to urge the plunger 134 axially away from the discharge end 110. Thus, the plunger 134 can be urged or activated for axial movement through the barrel by employing a knob 136 that is operatively connected (directly or indirectly) with the plunger accordingly. For instance, a user can push the knob 136 with the user's thumb while holding a handle or flange 142 with the user's index finger and middle finger (e.g., similar to using a syringe). Other means (e.g., trigger, spring activated, threads, etc.) can be employed for moving the plunger 134 in a desired direction. Additionally, after discharging the prosthesis 104 from the barrel 106, the plunger 134 may be removed so that the barrel provides a passage through which a corresponding implantation site (near the end 110) and the implanted prosthesis can be accessed.

Those skilled in the art will understand and appreciate various types of prostheses and implantable devices 104 that can be implanted via an implanter/introducer combination 100, 102. The implantable article 104 can have a fixed cross-sectional dimension, as shown by a solid line at 104. Alternatively, the device can be expandable to an enlarged cross-sectional dimension, such as indicated by dashed line at 104′.

By way of further example, to facilitate insertion of an expandable type of the article 104′ into the barrel 106, the prosthesis or device should be deformed to a reduced cross-sectional dimension, such as about less than an internal dimension of the barrel 106. As an example, the inner diameter of the barrel 106 can range from about 5 mm to about 15 mm, whereas the outer diameter of the article 104′ (in its expanded condition) might range from about 15 mm to about 35 mm. Thus, the barrel 106 can accommodate a prosthesis 102, which has been deformed to reduce cross-sectional dimension, without compromising the durability and operation of the article 104. Alternatively, as mentioned above the inner diameter of the barrel 106 can approximate the outer diameter of the prosthesis 104, such as when the prosthesis does not easily compress.

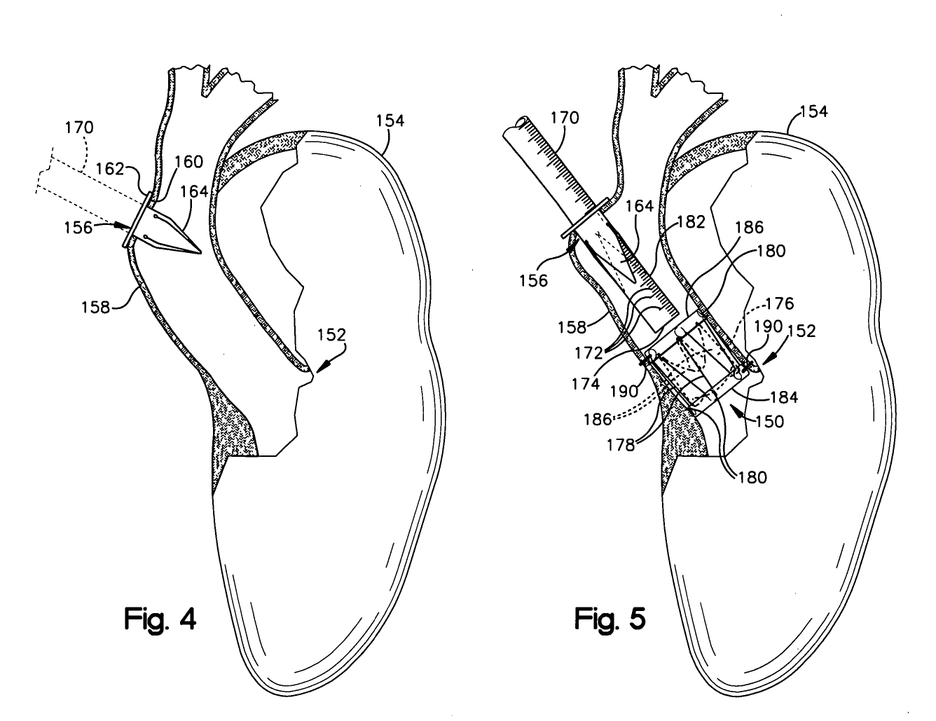

FIGS. 4 and 5 depict and example of a portion of a procedure that can be utilized for implanting a heart valve prosthesis 150 at an aortic annulus 152 of a patient's heart 154. In the example of FIGS. 4 and 5 it is assumed that at least portions of a defective valve or the entirely defective valve that is being replaced has been removed from the aortic position. For example, optional sutures (not shown) may be applied (through the barrel) at a location near the inflow end 184 and/or at the outflow end 186. Those skilled in the art will understand and appreciate that some heart valves might alternatively be implanted while the defective valve remains intact. Additionally, as described herein, different types of implantable articles might be implanted in a similar way to improve operation of a defective valve.

In FIG. 4, an introducer apparatus 156 has been inserted into the aorta 158. As an example, an incision is made at a desired location in the aorta 158 of the patient's heart 154 and the introducer apparatus 156 is inserted into the incision, such that a distal side 160 of a flange 162 engages the exterior aortic wall 158. A purse string (not shown) can be applied around the insertion to mitigate bleeding by tightening the heart muscle tissue around the sidewall of the introducer apparatus 156. The introducer apparatus 156 can be inserted separately or in combination with the implanter 170 while the implanter is inserted partially into the introducer apparatus. Once the introducer apparatus has been inserted, as shown in FIG. 4, a distal end portion 164 of the introducer apparatus 156 extends into the aorta 158 in the closed condition. The introducer apparatus 162 can be constructed according to any of the types described herein, such as shown and described in FIGS. 1-2 or include any combination of the features of FIGS. 1-2. Those skilled in the art will understand and appreciate that differently sized and configurations of introducer apparatuses can be utilized, for example, depending upon the location in which such apparatuses are to be implanted and the dimensions and configuration of the implanter. Additionally, the introducer apparatus can be inserted into other anatomical structures, including other blood vessels (e.g., pulmonary artery), to provide access to an intended implantation site.

In the example of FIG. 4, the introducer apparatus 156 is inserted into a position such that a generally direct path can be provided from the introducer apparatus to the desired implantation site (e.g., the aortic annulus 152) for implanting the prosthesis 150. An O-ring or other means (not shown) can be applied to a sidewall of the introducer apparatus 162 to help hold the jaws closed during its insertion into the heart 154 as well as to inhibit passage of the barrel of the implanter through the distal end of the introducer. The path from the introducer apparatus 156 to the implantation site 152 can be substantially linear or it can provide a substantially curved or indirect path depending upon the type of implanter being utilized.

FIG. 5 depicts an example of the heart valve prosthesis 150 being implanted at the aortic annulus 152 by employing an implanter 170 in combination with the introducer apparatus 156. Those skilled in the art will understand and appreciate various types of implanters 170 that can be utilized in conjunction with an introducer apparatus based on the teachings contained herein. Furthermore, it will be appreciated that the dimensions and configurations of the introducer apparatus 156 can be adapted according to the dimensions and configuration of the implanter 170, or the dimensions and configurations of the implanter can be adapted according to the dimensions and configurations of the introducer apparatus.

As described herein, indicia 172 along the sidewall of the implanter 170 provide a means for determining a measure of the distance that an end 174 of the implanter has been inserted to within the aorta 158. Thus, the measurement information from the indicia 172 can facilitate discharging the prosthesis 150 from the implanter at a desired implantation site 152. The distance and location of the implantation site 152 can be ascertained by employing a number of techniques. For example, the end 174 of the implanter 170 can be positioned via ultrasonic or radiographic means, such as a cardiac 3-D echo performed before and/or during the implantation procedure.

It is to be appreciated that the prosthesis 150 may be implanted at the aortic annulus 152 during a conventional open chest procedure or during a closed chest procedure. Because the only incision is in the patient's aorta, the implantation can be performed during very short open chest surgery, for example, with reduced cardiopulmonary bypass when compared to conventional procedures.

However, it is to be understood and appreciated that if the patient has a calcified aortic valve, the patient typically will be put on cardiopulmonary bypass to remove the defective valve or at least calcified portions thereof and to implant the prosthesis 150. Advantageously, a prosthesis 150 in accordance with the present invention may still be implanted more effectively than many conventional approaches even when cardiopulmonary bypass is utilized.

In the example of FIG. 5, the heart valve prosthesis 150 is depicted as a valve that can be implanted without requiring sutures (e.g., referred to as a sutureless valve), although sutures can be used. The prosthesis 150 includes a heart valve 176 mounted within a support 178 that is formed of a material that helps maintain a generally cylindrical configuration for the implanted valve 176.

According to one aspect of the present invention, the support 178 can be formed a shape memory material, such as Nitinol. For example, the support 178 can be formed from a small cylindrical tube of the shape memory material, such as via a laser cutting (ablation) process in which the desired sinusoidal sidewall is cut from the tube. Associated spikes 180 can be formed as an integrated structure having a desired shape and size to extend generally outwardly and arcuately form the respective ends of the support 178. Additionally, ends of the spikes 180 can have tapered or sharpened tips (with or without barbs) to facilitate gripping surrounding tissue of the aorta 158 when implanted in a sutureless implantation. Additionally or alternatively, some sutures can be utilized in combination with the spikes to retain the prosthesis 150 at a desired position. For instance, sutures 190 can be applied externally to prosthesis 150 after its implantation or sutures can be applied internally, such as through a passage defined by the implanter 170.

When the support is formed of a shape memory material, such as Nitinol, the support and prosthesis can be reduced to a reduced cross-section for insertion into a barrel 182 of the implanter 170. For instance, the support 178 can be heated to its transformation temperature and forced to a desired cross-sectional dimension and configuration (austenitic) form, corresponding to an expanded configuration of the support. The support 178 can then be bent or deformed to a reduced cross-sectional dimension when in its low-temperature (martensitic) form to facilitate its mounting the prosthesis 150 within a barrel 182 of the implanter 170, for example. When the prosthesis 150 is implanted and discharged from the barrel 182, the support 178 returns to its austenitic form, such as shown in FIG. 5.

The valve 176 can be a biological valve or a mechanical valve. For the example of a biological type of valve 176, the valve can be a homograft or xenograft, or it can be manufactured from a biological tissue material to include one or more leaflets arranged for providing substantially unidirectional flow of blood through the valve. The valve 176 includes an inflow end 184 and an outflow end 186 at axially opposed ends of the valve, with a sidewall portion extending between the ends thereof. The inflow end 184 of the valve 176 is positioned near a corresponding inflow end of the support 178. The prosthesis 150 can also include sidewall portion, which can be a tubular valve wall, such as for a homograft or xenograft valve 176. A plurality of leaflets 186 extend radially inward from the valve wall and coapt along their adjacent edges to provide for substantially unidirectional flow of blood through the valve 176. The valve 176 can be connected within the support 178 via sutures or other known connecting means, for example.

For example, when being implanted at an aortic position, an aortic valve (e.g., equine, porcine, bovine, etc.) can be utilized for the valve portion 176 of the prosthesis 150, although other types of valves could also be used. It is to be understood and appreciated that various types of valve 176 configurations of could be employed to provide the heart valve prosthesis 150 in accordance with an aspect of the present invention.

As another example, the valve 176 can include one or more leaflets mounted within a length of tubular valve wall or other generally cylindrical biocompatible material and operate in a known manner to provide for the unidirectional flow of fluid through the valve from the inflow to outflow ends. Examples of prostheses and valve includes those shown and described in U.S. Pat. Nos. 5,935,163, 5861,028 or 5,855,602, as well as others mentioned herein or otherwise known in the art. Those skilled in the art will further understand and appreciate that unstented as well as stented valves can be implanted by employing an introducer apparatus/implanter combination.

By way of further example, the valve 176 and any other biological material employed to provide the prosthesis 150 can be formed from natural biological material, such as a natural tissue sheets (e.g., animal pericardium, dura matter and the like), although other natural or synthetic biocompatible materials (e.g., molded collagen) also could be utilized. For instance, the biological materials can be cross-linked with glutaraldehyde and undergo a detoxification process with heparin bonding, such as according to a NO-REACT® treatment process available from Shelhigh, Inc. of Union, N.J. The NO-REACT® treatment improves biocompatibility of the valve 176 and mitigates calcification and thrombus formation.

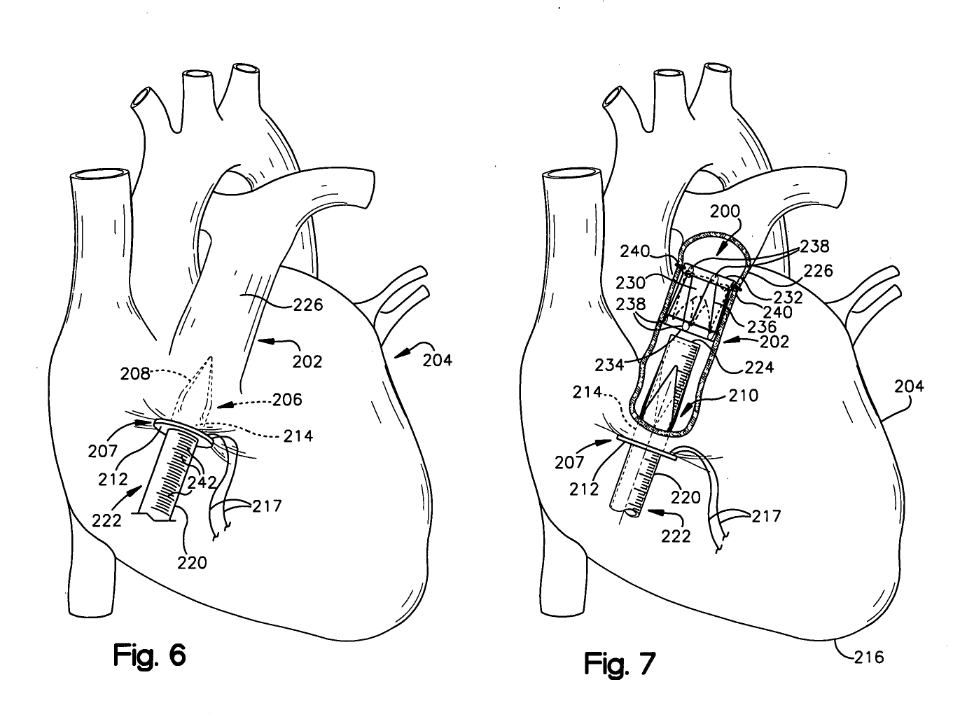

FIGS. 6 and 7 depict another example approach that can be utilized for implanting a heart valve prosthesis 200 according to an aspect of the present invention. In the example of FIGS. 6 and 7, the prosthesis 200 is implanted at a pulmonic position 202 of a patient's heart 204. Identical reference numbers are used in FIGS. 6 and 7 to identify corresponding features.

In the example of FIG. 6, an introducer apparatus 206 has been inserted into an anterior wall 207 of the right ventricle 210 (FIG. 7) of the heart 204, such that a distal end portion 208 of the introducer apparatus extends into the right ventricle of the heart. The insertion location into the heart 204 can vary to provide a substantially direct path to the implantation site at the pulmonic position 202. A flange portion 212 of the introducer apparatus 206 engages the exterior surface of the anterior wall 207 of the right ventricle. An intermediate portion 214 of the introducer apparatus 206 thus is surrounded by the heart muscle 204 at the anterior wall 207 of the heart. The engagement of the surrounding heart muscle 204 with the introducer apparatus 206 can be facilitated, for example, by applying a double purse string suture 217 generally around the insertion location, such as prior to making an incision through the heart 204 into the right ventricle.

The distal end portion 208 is in the closed condition during insertion of the introducer apparatus 206 into the heart muscle 204. The closed condition of the distal end portion 208 can be facilitated by employing an ring or other structure, such as shown and described in FIGS. 1-2. The location in which the introducer apparatus 206 is inserted into the heart 204 generally will depend on the type of implanter and the location of the implantation site.

In the example of FIGS. 6 and 7, a barrel 220 of an implanter 222 is depicted as being inserted into the introducer apparatus 206 and into the heart 204. The barrel 220 is substantially linear barrel, such as part of a catheter system or other type of implanter described herein. Those skilled in the art will understand and appreciate that a flexible and/or curved barrel 220 can be utilized. The barrel 220 is inserted at a position to provide a substantially linear or direct path for implanting the heart valve prosthesis at the desired pulmonic position 202. In FIG. 7, a distal end 224 of the barrel 220 has been inserted through the introducer apparatus 206 that so that respective members of the distal end portion 208 are urged apart to the open condition by the barrel 220 and thereby permit substantially free movement of the barrel through the introducer apparatus.

By inserting the introducer apparatus 206 through the anterior wall 207 proximal the pulmonary artery 226, a substantially direct or generally linear implantation of the prosthesis 200 can be performed with little or no cardiopulmonary bypass. As mentioned above, however, when the patient's defective valve is calcified, cardiopulmonary bypass will typically be performed for removing the calcified valve portions prior to implantation. The removing of the valve can be performed, for example, by employing a trocar or other tube inserted through the introducer apparatus 206 or during an open heart procedure prior to implanting the prosthesis 200.

Those skilled in the art will understand and appreciate other possible paths through the heart or associated arteries or veins that could be employed for positioning the distal end 224 of the barrel 220 to enable implantation of the prosthesis 200 in accordance with an aspect of the present invention.

The barrel 220 further can include indicia 242, such as corresponding to ruler markings, for providing an indication of measurement as to the distance which the end 224 of the barrel has been inserted into the heart 204. Accordingly, the measurement from the indicia 242 enables a user to discharge the prosthesis 200 into the pulmonary artery 226 at the desired implantation site, namely at the pulmonic position 202. Once at the desired position, the prosthesis 200 can discharged or ejected from the barrel 220 into the outflow of the right ventricle 210, as illustrated in FIG. 7.

The prosthesis 200 can be substantially similar to that, shown and described with respect to FIG. 5, although other types of valves can also be utilized. Briefly stated, the prosthesis 200 includes a valve 230 having an inflow end 232 and an outflow end 234 spaced therefrom. The valve 230 can be a natural tissue heart valve, such as a homograft or xenograft, although other types of biological tissue manufactured heart valves also could be utilized. Still further, the valve 230 can be a mechanical or biomechanical type of valve. The valve 230 can be mounted within a support or stent 236, such as one of the types described herein, or the valve could be unstented. Since the prosthesis 200 is implanted at the pulmonic position, which is exposed to lower blood pressure, a more pliant valve 230 generally can be utilized as compared to when implanting a prosthesis at the aortic position (FIG. 5).

For a sutureless type of implantation, the support 236 can include spikes or protruding portions 238 for engaging surrounding tissue of the pulmonary artery 226 in its implanted position. The spikes or protruding portions 238 thus inhibit axial and/or angular movement of the implanted prosthesis 200. While the spikes or protruding portions 238 are depicted as extending generally arcuately from the respective end portions, it will be appreciated that suitable spikes or protruding portions can be located at any external location of the support 236. It will be appreciated, however, that sutures can also or alternatively be employed as a means to secure the valve relative to the pulmonary artery 226. For instance, sutures 240 can be applied externally to prosthesis 200 after its implantation, or sutures can be applied internally, such as through a passage defined by the barrel 220.

While the foregoing examples illustrate implanting heart valve prosthesis in the heart of a patient, those skilled in the art will understand and appreciate that the introducer/implanter combination can be utilized to implant other types of implantable cardiac articles, such as annuloplasty rings, stents, as well as other devices. Additionally, the barrel of an implanter can be utilized as a trocar for performing surgical operations within the heart as may be required to facilitate adjustments of a heart valve prosthesis that has been discharged from an implanter or for adjusting the position of the heart valve prosthesis or other implanted device after its implantation. Additionally, those skilled in the art will understand and appreciate that the introducer apparatus and an implanter of sorts can be utilized for implanting other types of prosthesis and implantable devices associated with other anatomical structures or organs of the patient.

What has been described above includes examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. An introducer apparatus, comprising:
a flange;
a sleeve having a sidewall that extends longitudinally from the flange and terminates in a distal end portion; and
at least one adjustable opening operatively associated with the distal end portion, the adjustable opening being moveable between a substantially closed condition and an open condition, the open condition permitting substantially free movement of an elongate member axially through a passage defined by the sidewall.

2. The apparatus of claim 1, wherein the distal end portion of the sleeve further comprises at least one member moveable between the substantially closed condition and the open condition.

3. The apparatus of claim 2, wherein the at least one member further comprises at least two jaw members generally radially moveable relative to a central axis extending through the apparatus and moveable relative to each other between the substantially closed condition and the open condition.

4. The apparatus of claim 3, further comprising longitudinal slits extending through the sidewall to define the at least two jaw members when in the closed condition.

5. The apparatus of claim 4, wherein each of the longitudinal slits extends from a first axial position proximal the flange and intersect with at least one other of the slits near an end of the distal end portion.

6. The apparatus of claim 5, further comprising at least one aperture that extends through the sidewall of the sleeve at the first axial position and intersects with at least one of the longitudinal slits to facilitate movement of the at least two jaw members.

7. The apparatus of claim 3, wherein the distal end portion of the sleeve has a generally conical shape in the closed condition, with each of the jaw members providing a conical section according to the number of the at least two jaws.

8. The apparatus of claim 1, wherein the distal end portion of the sleeve has a generally conical shape in the closed condition tapering from a first cross-sectional dimension proximal the flange to a smaller cross-sectional dimension distal the flange.

9. The apparatus of claim 1, further comprising at least one structure operative to apply a radially inward force that inhibits movement of the elongate member axially through the passage defined by the sidewall.

10. The apparatus of claim 1, further comprising at least one structure applied to the sidewall to inhibit movement of the opening from the substantially closed condition to the open condition.

11. An implanter in combination with the introducer apparatus of claim 1, the implanter including a barrel terminating in an open end for discharging an article from an interior of the barrel through the open end, the introducer mounted substantially around at least a portion the barrel for axial movement along an external part of the barrel.

12. The combination of claim 11, further comprising indicia along the barrel, the flange aligning with the indicia to provide a measure of distance from the flange to the open end of the barrel.

13. The combination of claim 11, further comprising a biocompatible article located within the barrel of the implanter.

14. The combination of claim 13, wherein the article is expandable from a reduced cross-section, while located within the barrel, to an expanded cross-section after being discharged from the barrel.

15. The combination of claim 13, wherein the article further comprises a cardiac prosthesis.

16. The combination of claim 15, wherein the cardiac prosthesis further comprises a heart valve prosthesis.

17. An implantation system comprising:
an elongate barrel that terminates in an open end; and
an introducer apparatus having a first end spaced apart from a distal end by an elongated sidewall portion, the sidewall portion providing a passage that extends axially through the introducer apparatus, the passage being dimensioned and configured for receiving the barrel, the sidewall portion including a tapered distal end portion that is moveable from a substantially closed condition to an open condition in response to axial movement of the barrel from the first end and at least partially through the distal end such that barrel engages an interior of the distal end portion.

18. The system of claim 17, wherein the introducer apparatus further comprises a flange located at the first end.

19. The system of claim 18, wherein the flange comprises a substantially annular flange that extends from the first end of the introducer apparatus substantially transverse to the sidewall portion, an inner periphery of the flange defining an opening into the passage.

20. The system of claim 17, wherein the distal end portion further comprises at least two jaw members generally radially moveable relative to the axis, the at least two jaw members being moveable relative to the axis and to each other between the substantially closed condition and the open condition.

21. The system of claim 20, further comprising longitudinal slits extending through the sidewall portion when in the closed condition to define adjacent side edges of each of the at least two jaw members.

22. The system of claim 21, wherein each of the longitudinal slits extends from about a first axial position of the sidewall portion and intersects with at least one other of the longitudinal slits near an end of the distal end portion.

23. The system of claim 22, further comprising at least one aperture that extends through the sidewall portion at an axial position intermediate the first end and the distal end of the introducer apparatus, the at least one aperture intersecting at least one of the longitudinal slits to facilitate movement of the at least two jaw members between the closed condition and the open condition.

24. The system of claim 20, wherein the distal end portion has a generally conical shape in the closed condition tapering from a first cross-sectional dimension proximal the first end of the introducer apparatus to a smaller cross-sectional dimension at the distal end of the introducer apparatus, each of the jaw members attributing to a portion of the conical shape according to the number of the at least two jaws.

25. The system of claim 17, further comprising indicia along the barrel, the first end of the introducer apparatus aligning with the indicia to provide a measure of distance from the first end of the introducer apparatus relative to the open end of the barrel.

26. The system of claim 17, further comprising an implanter, the barrel corresponding to a distal end portion of the implanter at least a portion of which is moveable through the introducer apparatus, the implanter being configured to discharge an implantable article from a location within the barrel through the open end of the barrel.

27. The system of claim 26, further comprising a substantially biocompatible article located within the barrel of the implanter.

28. The system of claim 27, wherein the article is expandable from a reduced cross-section to an expanded cross-section after being discharged from within barrel.

29. The combination of claim 27, wherein the article further comprises a cardiac prosthesis.

30. The apparatus of claim 17, further comprising at least one structure operative to apply a radially inward force to the introducer apparatus to inhibit movement of the elongate member axially through the passage defined by the sidewall of the introducer apparatus.

31. The apparatus of claim 17, further comprising at least one structure applied to the sidewall of the introducer apparatus to inhibit movement of the tapered distal end portion from the substantially closed condition to the open condition.

32. An implantation system comprising:
means for implanting an article;
means for providing a passage through an opening formed in tissue of a patient to guide the means for implanting toward an implantation site, the means for providing having an end portion that is moveable from a closed condition to an open condition in response to axial movement of the means for implanting through the means for providing to facilitate positioning an end of the means for implanting near the implantation site; and
means for discharging the article from the means for implanting.

33. The system of claim 32, further comprising means for inhibiting movement of the means for implanting axially through the passage.

34. The system of claim 32, further comprising means for inhibiting movement of the end portion to the open condition.
